(19) United States
(12) Patent Application Publication
(10) Pub. No.: US 2025/0147027 A1
(43) Pub. Date: May 8, 2025

(54) PROSTATE CANCER DETECTION REAGENT AND USE THEREOF IN PROSTATE CANCER DETECTION

(71) Applicants: JIANGSU XIANSIDA BIOTECHNOLOGY CO., LTD., Taizhou City, Jiangsu Province (CN); XIANSIDA NANJING BIOTECHNOLOGY CO., LTD., Nanjing, Jiangsu Province (CN)

(72) Inventors: Cuiying CHEN, Nanjing City, Jiangsu Province (CN); Lei WANG, Nanjing City, Jiangsu Province (CN); Zongnan TAN, Nanjing City, Jiangsu Province (CN)

(73) Assignees: JIANGSU XIANSIDA BIOTECHNOLOGY CO., LTD., Taizhou City, Jiangsu Province (CN); XIANSIDA NANJING BIOTECHNOLOGY CO., LTD., Nanjing, Jiangsu Province (CN)

(21) Appl. No.: 18/691,059

(22) PCT Filed: Sep. 14, 2022
(86) PCT No.: PCT/CN2022/118801

§ 371 Date: Mar. 12, 2024

(30) Foreign Application Priority Data
Sep. 15, 2021 (CN) 202111079840.8

(51) Int. Cl. G01N 33/574; G01N 33/58

(52) U.S. Cl. CPC G01N 33/57434; G01N 33/582

(57) ABSTRACT

The invention provides a prostate cancer detection reagent and a preparation method thereof. The detection reagent is prepared by mixing the following reagents, reagent A is prepared by adding SDS with a mass concentration of 0.5˜5% into ammonium bicarbonate solution with a concentration of 10 mM; Reagent B was prepared by mixing 0.01-10 U/10 μL glycoamidase and 0.01-10 U/10 μL Sialidase, and the pH value of the mixed solution was 4-9. Reagent C: Prepared from 8-Aminopyrene-1,3,6-trisulfonic acid trisodium salt dissolved in DMSO, the concentration is 0.01 mM˜1 M; Reagent D: Stopping solution. The invention determines the N-glycan profile in serum by the detection reagent, quantifies the peak value for statistical analysis, and provides a method for establishing a model of the N-glycan profile in serum of prostate cancer to detect prostate cancer.

### TECHNICAL FIELD

The invention belongs to the field of biomedicine technology and involves a method for detecting prostate cancer, in particular a detection method for prostate cancer based on the spectrum of serum glycoprotein oligosaccharide detection (G-Test).

### BACKGROUND TECHNIQUE

Prostate cancer is an epithelial malignant tumor occurring in the prostate, and it is the most common malignant tumor of the male genitourinary system. Prostate cancer progresses particularly slowly, making it difficult to detect in its early stages. The main clinical manifestations of patients include urinary difficulties, lumbago, urgent urination, frequent urination, urination pain, and other urinary tract symptoms. Treatment primarily involves radical resection, surgery, or medical castration. Early-stage prostate cancer can be cured, while conservative treatment is preferred for advanced cases.

The cause of prostate cancer is related to genetics, environment, food, and age, there is a family history of prostate cancer, the incidence is relatively high, and the age of onset will be younger. Prostate cancer tends to occur in older men over the age of 65, people with unhealthy lifestyles, and people who have had prostate cancer in their immediate family, and factors such as diet and obesity are easily induced.

In terms of epidemiology, the incidence of prostate cancer 10 years ago in our country was about 0.005%˜0.006%, and the growth rate in the last 10 years in our country is relatively fast. In the past, prostate cancer was a highly common disease in Western countries, especially in the United States, where the incidence of men ranked first and the mortality rate ranked second. The incidence of prostate cancer continues to rise globally, with nearly 1.3 million new cases and 359,000 deaths worldwide in 2018. The incidence of malignant tumors in men is much higher than the mortality rate, and the risk of prostate cancer increases with age.

At present, the most important auxiliary diagnosis for prostate cancer patients is prostate-specific antigen (PSA) detection; However, this method is not sensitive to early prostate cancer, and when it is found, it is basically in the middle and late stages, and the prognosis is poor. The other way is biopsy for pathological anatomy; Of these two ways, the former is not sensitive, and the latter is more traumatic.

Glycosylation is the most common post-translational modification of protein. Glycosylation is a process in which glycans are transferred to proteins and special amino acid residues on proteins to form glycoside bonds under the action of glycosyltransferase. Most glycoproteins are secreted proteins and are widely present in cell membranes, interstitial cells, plasma, and mucus. Some enzymes and hormones are glycoproteins. Glycoproteins have many biological functions. Some glycoproteins such as procollagen are structural proteins. Many enzymes and hormones (such as luteinizing hormone, thyrotropin, etc.) have a glycoprotein structure, and many glycoproteins in the blood are responsible for the transport of inorganic ions (Fe2+, Ca2+, Cu2+, etc.) and hormones and other bioactive substances, blood coagulation (fibrinogen is a glycoprotein) and antibody activity. Lectins have the ability to agglutinate cells, and the oligosaccharide can also play the role of stabilizing the peptide chain. Another important function of glycoproteins is to participate in various recognition phenomena on the cell surface directly or indirectly. Because of the importance of the glycan in the glycoprotein to maintain the biological function of the body, the change of the glycan can help to elucidate the molecular mechanism of abnormal biological behavior such as inflammation, invasion, and metastasis of tumor cells to surrounding tissues. At present, changes in the N-glycan have been found in a variety of tumors.

oligosaccharide is an important biological information molecule, which plays a unique role in many physiological and pathological processes. The structure of oligosaccharides is very complex and exhibits microscopic heterogeneity, and its analysis and structural analysis have been the bottleneck of glycobiology research. At present, the analysis methods of oligosaccharide structure have developed rapidly, mainly including (1) high-performance liquid chromatography (HPLC): High resolution, fast detection speed, high repeatability, HPLC columns can be used repeatedly, but the column efficiency will be reduced with the increase of the number of uses, and the mobile phase is toxic, equipment operation requires highly trained professionals and the equipment is relatively expensive, and the solvent needs to be strictly purified; (2) Mass spectrometry (MS): MS has the advantages of high sensitivity, can obtain a variety of structural information and suitable for analysis of mixtures, and is an ideal means of qualitative and quantitative analysis of oligosaccharide. However, MS is precise, the equipment operation is complex, and the mass spectrometer is expensive, which is not suitable for clinical popularization and use; (3) Capillary electrophoresis (CE): CE has low cost, high column efficiency, high sensitivity, fast speed, low injection volume and simple operation, but the repeatability is not high, and the stability is not as good as HPLC.

G-Test (Glycan-Test) is a DNA analyzer-based capillary microelectrophoresis technology (DSA-FACE). The N-glycan of glycoproteins in prostate fluid samples is fluorescence-labeled and then separated by capillary microelectrophoresis. The content of N-glycan obtained by measuring the fluorescence signal is the fingerprint (referred to as the G-Test). The detection technology has the advantages of high sensitivity, simple operation, small amount (2 μL serum), high repeatability, good stability, high throughput (96-well plate), and other oligosaccharide analysis technology can not be compared, which is suitable for general laboratory departments, and is expected to be used in clinical promotion.

### Content of Invention

The purpose of the invention is to provide a prostate cancer monitoring reagent, through which the serum glycan profile is measured, the peak value is quantified, and statistical analysis is carried out, thus providing a method for establishing the serum glycan profile model of prostate cancer.

The technical scheme of the invention is as follows:

A prostate cancer monitoring reagent consists of the following reagents:
Reagent A: The solution of ammonium bicarbonate with a concentration of 10 mM is prepared by adding SDS with a mass concentration of 0.5˜5%;
Reagent B: It is prepared by mixing 0.01-10 U/10 μL glycoamidase and 0.01-10 U/10 μL Sialidase, and the pH value of the mixed solution is 4˜9;
Reagent C: It is prepared from 8-Aminopyrene-1,3,6-trisulfonic acid trisodium salt dissolved in DMSO, the concentration is 0.01 mM˜1 M;
Reagent D: Stopping solution.

Preferably, the volume ratio of reagent A, reagent B, and reagent C is 2:2:1.

A method for preparing a prostate cancer detection reagent comprises the following steps:

### Step 1 Preparation of Oligosaccharide

4 μL of reagent A was added to the inactivated 2 μL serum sample, denatured, cooled to room temperature, added 4 μL of reagent B, and incubated for 1˜6 h;

### Step 2 Labeling of Oligosaccharide

2 μL of reagent C was added to the liquid obtained in step 1 for fluorescence labeling, and then 150 μL of reagent D was added to terminate the labeling reaction;

### Step 3 Oligosaccharide Separation Analysis

Take 10 μL of the liquid treated in step 2, use the analyzer to separate the oligosaccharide, and get the spectrum.

Preferably, the denaturation temperature in the preparation of step 1 oligosaccharides is not less than 75° C. for heating, and the incubation temperature is not less than 25° C.

Preferably, the fluorescently labeled temperature in step 2 is 50 to 90° C.

Application of a composition in the preparation of prostate cancer monitoring reagents, the composition detects prostate cancer by the ratio of NA2/NA2F.

Glycosylation refers to the formation of N-glycan through complex interactions between hundreds of enzymes, transcription factors, ion channels, and proteins, and is involved in a variety of molecular processes, such as protein folding, cell adhesion, molecular transfer, signal transduction, and regulation of receptor activity. Changes in N-glycan on glycoproteins are associated with disease.

A large number of previous studies have found that the relative contents of N-oligosaccharide NA2(bigalacto biantennary glycan) and NA2F(bigalacto core-alpha-1,6-fucosylated bisecting biantennary glycan) in serum of prostate cancer patients have significantly changed compared with that in normal serum, so they can be used as markers for detecting prostate cancer.

Given the existing prostate cancer detection, the specificity and accuracy of blood detection and imaging detection are not high, the pathological examination is traumatic, prone to sampling errors, and patient dependence is poor, the invention provides a prostate cancer N-glycan detection method.

### Materials and Methods:

1. Test samples: serum from prostate cancer patients and healthy people.

2. Experimental equipment: oligosaccharide analyzer, PCR, centrifuge.

3. Reagent preparation:
Reagent A: The solution of ammonium bicarbonate with a concentration of 10 mM is prepared by adding SDS with a mass concentration of 0.5˜5%;
Reagent B: It is prepared by mixing 0.01-10 U/10 μL glycoamidase and 0.01-10 U/10 μL Sialidase, and the pH value of the mixed solution is 4˜9;
Reagent C: It is prepared from 8-Aminopyrene-1,3,6-trisulfonic acid trisodium salt dissolved in DMSO, the concentration is 0.01 mM˜1 M;
Reagent D: Stopping solution.

4. oligosaccharide sequencing test steps:

### Step 1 Preparation of Oligosaccharide

4 μL of reagent A was added to the inactivated 2 μL serum sample, denatured, cooled to room temperature, added 4 μL of reagent B, and incubated for 1˜6 h;

### Step 2 Labeling of Oligosaccharide

2 μL of reagent C was added to the liquid obtained in step 1 for fluorescence labeling, and then 150 μL of reagent D was added to terminate the labeling reaction;

### Step 3 Oligosaccharide Separation Analysis

Take 10 μL of the liquid treated in step 2, use the analyzer to separate the oligosaccharide, and get the spectrum.

### 5. Monitoring Contrastive Analysis

Peak quantization was performed on the obtained N-glycan, and the relative content of each peak was quantitatively calculated by comparing the peak height value of each peak with the sum of the heights of all peaks. The composition was further statistically analyzed by the function NA2/NA2F to detect prostate cancer.

Compared with the existing technology, the invention has the following beneficial effects:
(1) The method of the invention is based on the fingerprint of detecting N-glycan in serum glycoprotein, creating a detection method and detection system for prostate cancer diagnosis. This method can allow many highly suspected prostate cancer patients to receive routine, non-invasive detection, help doctors detect, and timely monitor the occurrence and progression of the disease, and has a more obvious accuracy than other current technologies, the sensitivity of prostate cancer detection reached 97.2%.
(2) Compared with the existing technology, the detection method of the invention has an accuracy of 97.2%, which is superior to the existing detection method. The reagent of the invention can enable many high-risk people to receive routine and non-invasive detection, help doctors and patients timely monitor the occurrence and progression of prostate cancer, and is expected to be widely used in clinical practice.

### ILLUSTRATED DESCRIPTION

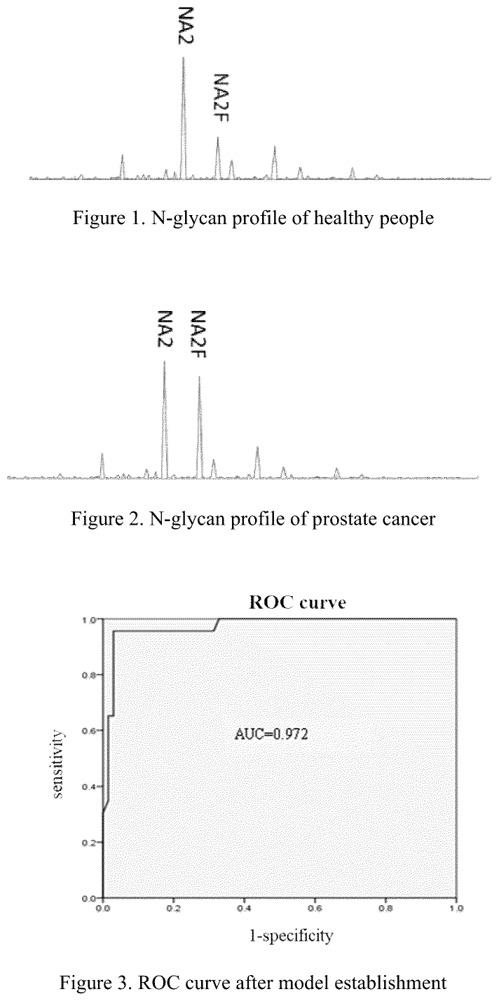

FIG. 1 shows the N-glycan profile in the healthy people group.

FIG. 2 shows the N-glycan of serum glycoprotein in prostate cancer. The abbreviations of oligosaccharides in the graph are NA2F, Bigalacto core-α-1, 6-fucosylated biantennary; NA2, Briantennary.

FIG. 3 shows the ROC curve after model establishment. The ROC curve used to identify prostate cancer was determined by the function NA2/NA2F. A total of 90 samples were detected, including 23 serum samples of prostate cancer and 67 samples of non-prostate cancer (prostatitis and prostatic hyperplasia). The AUC under the curve was 0.972.

### CONCRETE IMPLEMENTATION MODE

The present invention is further described below in combination with embodiments and drawings. It should be noted that the following embodiments are used only to illustrate and not to limit the scope of the invention. Experimental methods not specified in the following embodiments are usually tested under conventional conditions or conditions suggested by the manufacturer, with reagents for laboratory use.

### Example 1 Detection of Prostate Cancer

### Materials and Methods:

1. Test samples: serum from prostate cancer patients and healthy people.

2. Experimental equipment: capillary electrophoresis analyzer, PCR, centrifuge.

3. Reagent preparation:
Reagent A: The solution of ammonium bicarbonate with a concentration of 10 mM was prepared by adding SDS with a mass concentration of 0.5˜5%;
Reagent B: It is prepared by mixing 0.01-10 U/10 μL glycoamidase and 0.01-10 U/10 μL Sialidase, and the pH value of the mixed solution is 4˜9;
Reagent C: It is prepared from 8-Aminopyrene-1,3,6-trisulfonic acid trisodium salt dissolved in DMSO, the concentration is 0.01 mM˜1 M;
Reagent D: Stopping solution.

4. oligosaccharide sequencing test steps:

### Step 1 Preparation of Oligosaccharide

4 μL of reagent A was added to the inactivated 2 μL serum sample, denatured, cooled to room temperature, added 4 μL of reagent B, and incubated for 1˜6 h;

### Step 2 Labeling of Oligosaccharide

2 μL of reagent C was added to the liquid obtained in step 1 for fluorescence labeling, and then 150 μL of reagent D was added to terminate the labeling reaction;

### Step 3 Oligosaccharide Separation Analysis

Take 10 μL of the liquid treated in step 2, use the analyzer to separate the oligosaccharide, and get the spectrum.

### 5. Monitoring Contrastive Analysis

Serum samples collected from 90 patients with prostate cancer and healthy people were processed by the G-Test technique, including 23 with prostate cancer and 67 with non-prostate cancer (prostatitis and prostatic hyperplasia). The N-glycan profile obtained by the G-Test was analyzed statistically.

The peak height of each peak was divided by the sum of all peak heights, and the relative content of each peak was obtained by quantitative calculation, that is, the peak value of the N-glycan profile was quantified, and then the 9 oligosaccharide peaks in the N-glycan profile of the quantified prostate cancer group and the healthy people group were compared and statistically analyzed. As shown in FIG. 1 and FIG. 2, the N-glycan profile of human serum shows nearly 9 peaks of N-glycan, and the mobility of oligosaccharide chains varies according to the molecular size, that is, different peaks on the N-glycan profile represent different oligosaccharide, and the measured peak height represents the relative concentration of oligosaccharide. The N-glycan profile was further statistically analyzed by the ratio of NA2/NA2F to detect prostate cancer.

The ROC curve used to identify prostate cancer was determined by the function NA2/NA2F. The total number of samples was 90, including 23 serum samples of prostate cancer and 67 samples of non-prostate cancer (prostatitis and prostatic hyperplasia). The area under the curve AUC=0.972.

Compared with the existing technology, the detection method of the invention has an accuracy of 97.2%, which is superior to the existing detection method. The reagent of the invention can enable many high-risk people to receive routine and non-invasive detection, help doctors and patients timely monitor the occurrence and progression of prostate cancer, and is expected to be widely used in clinical practice.

The specific embodiments described above, combined with the accompanying drawings, provide further detailed explanations of the purpose, technical scheme, and beneficial effects of the invention. It should be understood that the above is only a specific embodiment of the invention, but not a limit on the scope of protection of the invention, and the technical personnel in the field should understand that within the spirit and principles of the invention. any modification, equivalent replacement, improvement, etc. that can be made without creative labor shall be included in the scope of protection of the invention.

1. A prostate cancer monitoring reagent characterized in that it comprises the following reagents:
Reagent A: The solution of ammonium bicarbonate with a concentration of 10 mM is prepared by adding SDS with a mass concentration of 0.5˜5%;
Reagent B: It is prepared by mixing 0.01-10 U/10 μL glycoamidase and 0.01-10 U/10 μL Sialidase, and the pH value of the mixed solution is 4˜9;
Reagent C: It is prepared from 8-Aminopyrene-1,3,6-trisulfonic acid trisodium salt dissolved in DMSO, the concentration is 0.01 mM˜1 M;
Reagent D: Stopping solution.

2. The prostate cancer detection reagent according to claim 1, characterized in that volume ratio of reagent A, reagent B, and reagent C is 2:2:1.

3. The method for preparing prostate cancer detection reagents according to claim 1, characterized by including the following steps:
Step 1 Preparation of oligosaccharide
4 μL of reagent A was added to the inactivated 2 μL serum sample, denatured, cooled to room temperature, added 4 μL of reagent B, and incubated for 1˜6 h;
Step 2 Labeling of oligosaccharide
2 μL of reagent C was added to the liquid obtained in step 1 for fluorescence labeling, and then 150 μL of reagent D was added to terminate the labeling reaction;
Step 3 Oligosaccharide separation analysis
Take 10 μL of the liquid treated in step 2, use the analyzer to separate the oligosaccharide, and get the spectrum.

4. The method for preparing prostate cancer detection reagent according to claim 3, characterized in that the denaturation temperature in preparation of oligosaccharide in step 1 is not less than 75° C. heating, and the incubation temperature is not less than 25° C.

5. The method for preparing prostate cancer detection reagent according to claim 3, characterized in that the temperature of the fluorescent label in step 2 is 50˜90° C.

6. (canceled)
